(19) United States
(12) Patent Application Publication
(10) Pub. No.: US 2006/0128953 A1
(43) Pub. Date: Jun. 15, 2006

(54) Crystal refining technologies by controlled crystallization

(76) Inventors: Yuping Shi, Madison, WI (US); Baomin Liang, Madison, WI (US); Richard W. Hartel, Madison, WI (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/284,531

(22) Filed: Nov. 21, 2005

Related U.S. Application Data
(60) Provisional application No. 60/634,615, filed on Dec. 9, 2004.

(51) Int. Cl. C07H 3/04

(52) U.S. Cl. 536/123.13

(57) ABSTRACT

A method is provided for making large, uniform and individual crystals from aqueous solutions including the steps of obtaining a concentrated aqueous solution by means of evaporation; rapidly cooling the solution from a post-evaporation high temperature to a first lower temperature, wherein the first lower temperature is lower than the post-evaporation high temperature and further wherein the first lower temperature is an isothermal crystallization temperature of said solution; generating a batch of initial nuclei by inducing nucleation at the first lower temperature and starting crystal growth; uniformly spreading the initial nuclei into a bulk solution; maintaining simultaneous and rapid growth of crystals from the nuclei at the first lower temperature for a predetermined length of time; continuing the growth of the crystals to produce large, uniform and individual crystals for a predetermined length of time at a temperature that varies gradually from between a first lower temperature to a second lower temperature, wherein the second lower temperature is a temperature lower than the first lower temperature and further wherein the second lower temperature is an end temperature of crystallization; and recovering the large, uniform and individual crystals. Parameters and a system for producing lactose monohydrate crystals using the method are also provided.

### RELATED APPLICATION INFORMATION

This application claims priority from U.S. Application No. 60/634,615, filed on Dec. 9, 2004, the contents of which are herein incorporated by reference.

This invention was made with United States government support awarded by the following agencies: USDA AGRIC-CSCREES Award No: 2001-35503-10815. The United States has certain rights in this invention.

### FIELD OF THE INVENTION

The present invention relates to improvement in crystal refining technologies, more particularly, the improvement of lactose refining by controlled crystallization.

### BACKGROUND

Lactose, a disaccharide comprised of glucose and galactose, is the main constituent of milk whey. Lactose production in the U.S. was estimated to be 440 million pounds in 2004 and was used in a large number of human and animal food products. Lactose production starts by the removal of cheese and whey cream from milk. The remaining whey is evaporated to concentrate lactose. Lactose crystals are formed in crystallization tanks by holding the slurry over a carefully controlled time and temperature profiles. After crystallization, the solid is washed in decanter centrifuges, dried and milled before packing. However, the current lactose production methods are not optimized for quality (purity, color, crystal size, etc.), recovery (washing loss), efficiency (batch operations and 24-27 hours of process time), capital cost and energy consumption.

Commercial lactose products are primarily manufactured from whey, an intermediate diary product obtained in cheese making. Approximately 9 pounds of whey are generated for each pound of cheese produced. Dry materials in whey take about 6% of the total weight and the rest is water. The composition of whey may vary depending on different milk sources and cheese productions. The content of the major components of whey is shown in Table 1.

TABLE 1
Content of major components in whey

| | Component | | | | |
|---|---|---|---|---|---|
| | Lactose | Protein | Ash | Fat | Others |
| % (dry basis) | 72-80 | 11-12 | 9-10 | 1 | 1 |

As one of the commonly used sugars, lactose is utilized in many areas. In the food industry, lactose is widely used as an ingredient in confectionary, beverage, infant foods, frozen foods, prepared foods, etc. In the pharmaceutical industry, lactose is used as additives for tabletting. In the chemical industry, lactose can be used as a basis for the production of lactulose, lactitol and lactobionic acid, etc. Lactose is also used for feed in the agricultural industry.

Chemically, commercial lactose is available in the most stable form, namely, crystalline α-lactose monohydrate. Depending on the different requirements of the application, lactose products have different grades: crude, food (edible), pharmaceutical, etc. The quality aspects for lactose mainly include purity (content of lactose, impurities and moisture), color, crystal size distribution (mean size and deviation), microorganism count, odor, etc. Generally speaking, products with high quality should have a higher content of lactose, fewer impurities, less moisture, a higher whiteness, a desired size with narrow distribution and fewer microorganisms.

Crystallization is the means to obtain commercial products of lactose from whey. However, to efficiently produce high quality α-lactose monohydrate, other operations combined with crystallization are necessary. A typical existing commercial process for refined lactose manufacture is briefly shown in FIG. 1.

Whey contains 11-12% (dry basis) of proteins that do not allow for good control of the crystallization and the quality of the lactose product. But on the other hand, proteins are valuable materials worth being recovered as other dairy products (whey protein isolate, etc.). FIG. 1 shows that in a lactose process, whey is treated by using nano-filtration and/or ultrafiltration to remove most of the proteins from the solution first, and then permeate is obtained.

Water is then evaporated from the obtained permeate by using vacuum evaporation at a higher temperature to concentrate the solution to a high level of about 60% total solids. The concentrated permeate will be a supersaturated solution for lactose refining at lower temperatures. It is transported by a pump into crystallizers (crystallizing tanks) equipped with a cooling jacket. Agitation is then applied in the crystallizers. As the crystallizing tanks are filled, the concentrated permeate is then cooled to a lower temperature at which it is supersaturated. Lactose crystals are then added as seeds in the concentrated permeate to generate nuclei of lactose. Crystallization (nucleation and crystal growth) proceeds as the temperature of the suspension is gradually decreased from about 80° C. to about 25° C. The obtained lactose crystal slurry is then centrifuged to separate the crystals from the mother solution, also referred to as the De-Lactose Permeate (DLP). The discharged cake from the centrifuge must then be washed by using water to remove the impurities, because the mother solution is entrained in the spaces between the lactose crystals. Washing is usually carried out in a washing tank. The wash solution will be disposed or recycled. After washing, the slurry is centrifuged to remove the wash solution and moist lactose crystals are obtained. Finally, drying is performed to remove moisture and a refined lactose product is obtained.

Crystallization is the most important operation step in the process and affects the efficiency of the process and the product quality. Crystallization, usually including nucleation and crystal growth, is a complex physico-chemical phenomenon of phase transition. Many factors including concentration, temperature, viscosity of solution, agitation intensity, etc., have an impact on crystallization. To efficiently obtain high quality lactose, crystallization must be optimally controlled. Generally speaking, well-developed, large lactose crystals with narrow crystal size distribution (CSD) will have the least mother solution entrainment and are easily washed, leading to high quality.

However, the methods used in the crystallization processes currently known in the art are far from optimization. In these currently existing processes, the filling of the crystallizer takes about 6 hours. Cooling and crystallization last for 14-18 hours. Therefore, the currently existing processes take 20-24 hours for crystallization. The crystallization procedures and the temperature profile of the existing process are illustrated in FIG. 2. A practical profile for the temperature, concentration and supersaturation of the solution in the currently existing processes is schematically shown in FIG. 3. Cooling is carried out gradually from about 80° C. to about 25° C. in order to reach lactose yield as high as possible. Under these conditions, supersaturation exists and agitation is applied throughout the process. Nucleation and crystal growth occur throughout the entire duration. As crystallization proceeds, the concentration of the solution gets lower. Correspondingly, the supersaturation in the system is changed according to the concentration and solubility at a certain temperature. However, since the growth of crystals is accompanied by secondary nucleation throughout this process, the resulting crystals are in a very wide range of size with, a lot of small crystals as shown in FIG. 4. These small crystals plus a wide size distribution cause crystal aggregation and large solution entrainment that make centrifuging separation and washing difficult. This results in low quality, large loss of lactose and low efficiency. All these increase energy consumption and capital cost for large equipment, particularly the crystallizers.

Van den Bos (“Background of Technologies used for the Production of Lactose”, Chapter 15, Session IV, Bulletin of the IDF—212, pp. 99-102, 1987) discloses large scale production of lactose from whey. Two continuous processes having five major steps for lactose manufacture from whey or whey permeate are compared. The method described increases the crystallization time to 8-12 hours. Although the production yield is increased, the production time is lengthened according to the disclosed processes.

U.S. Pat. No. 3,721,585 discloses a method for manufacturing lactose crystals from raw whey. Acid is added to the raw whey and then concentrated by evaporation. The concentrate is then commingled with lactose crystals at a temperature range from 80° F. to 120° F. and agitated. During the crystal growth period, the contents in the crystallization tank are cooled from 60° F. to 90° F. over a period of from 12 hours to 24 hours. Needle-shaped lactose crystals are then harvested, centrifuged, washed and dried.

U.S. Pat. No. 4,404,038 discloses a method for manufacturing lactose crystals by continuously cycling lactoserum. The lactoserum is used to feed the first phase of crystallization and heated to a temperature of from 50° C. to 55° C. The lactoserum is then deproteinized or demineralized at a temperature of from 65° C. to 70° C. Crystal seeds are continuously fed into the crystallization apparatus. During the crystal growth period, a mother liquor which has a temperature of from 50° C. to 70° C. is cooled to a temperature of from 10° C. to 15° C. The resultant crystals have a size between 50 to 250 microns.

U.S. Pat. No. 4,955,363 discloses a method for manufacturing lactose crystals with chromatography separation. Whey concentrate is cooled from 75° C. to 15° C. at a rate of 2° C. per hour. The resulting mother liquor is then purified by heating it to about 60° C. to 70° C. using chromatography separation.

U.S. Patent No. 6,140,520 discloses a continuous crystallization system with controlled nucleation for milk fat fractionalization. The disclosed apparatus and method are for fractionating mixed triglycerides, more particularly for anhydrous milk fat. The disclosed apparatus and method focus on the uniform maximum melt temperature of the solid fraction.

European Patent Application No. 0,249,368 A2 discloses a method for isolating lactose from whey. The disclosed process focuses on the demineralization of whey and the crystallization steps uses conventional techniques and apparatus.

Therefore, there is a need for a method and an apparatus for making large, uniform crystals having a narrow size distribution that is optimal, particularly for lactose monohydrate crystals.

### SUMMARY OF THE INVENTION

This invention relates to methods and systems for improving crystallization, and more particularly, the crystallization of lactose monohydrate. More specifically, the methods and systems according to the present invention produce larger, uniform and individual crystals having a narrow crystal size distribution while aggregation of secondary nuclei is avoided. Even though the methods and systems of the present invention can be used to produce larger, uniform and individual crystals having a narrow crystal size distribution from other raw materials, a preferred embodiment of the present invention comprises the methods and systems for improving the crystallization of lactose monohydrate.

Although crystallization is improved by the methods and systems according to the present invention by controlling temperature, other parameters and steps are also carefully controlled in order to produce larger, uniform and individual crystals having a narrow crystal size distribution (CSD) as described herein.

The method of the present invention involves a number of steps. Specifically, when the raw material is whey permeate for growth of lactose crystals, concentrated permeate is first rapidly cooled to an optimal temperature for nucleation and subsequent crystal growth. Then, a batch of nuclei with suitable numbers is generated by controlled, instantaneous, induced nucleation, which takes a very short time. The generated nuclei are allowed to grow isothermally at about 40° C. to about 55° C., and preferably about 50° C., rapidly and simultaneously under optimal conditions (temperature, viscosity, well-suspended, etc.) for about 1 hour. The crystals are then further developed to reach a maximum yield by gradual cooling to about 30° C. or lower in about 1 to about 2 hours. The lactose crystals produced by the methods of the present invention are non-aggregated crystals having a large size and a narrow size distribution. The resulting crystals are high in quality for lactose crystalline products because 1) the nuclei are generated in one batch and grow simultaneously throughout the process, 2) secondary nucleation is avoided and no or very limited secondary nuclei are generated, 3) the crystals grow at a maximum rate under the optimal operating conditions, and 4) aggregation of crystals is avoided. Since the lactose crystals of the present invention have a good CSD, it is much easier for filtration and/or centrifuging of the slurry, washing off the impurities from the crystals and drying of the moist lactose. As a result, the production operations are more efficient.

The size of major equipment used in the present invention, the crystallizer, is about ⅕ to about 1/10 of that for existing processes. The energy consumption of the methods according to the present invention is much lower than those of current processes due to the much higher efficiency (about 3 hours versus about 24 hours for batch operation). In addition, the methods of the present invention are employed in flexible manners such as in a batch operation, in a continuous operation or a combination thereof.

The method and system according to the present invention are applicable for growing any crystal from an aqueous solution of different raw materials. Accordingly, one object of the present invention is accomplished by providing a method for making large, uniform and individual crystals from aqueous solutions that includes the steps of:

a) obtaining a concentrated aqueous solution by means of evaporation;

b) rapidly cooling said solution from a post-evaporation high temperature to a first lower temperature, wherein the first lower temperature is lower than the post-evaporation high temperature and further wherein the first lower temperature is an isothermal crystallization temperature of said solution;

c) generating a batch of initial nuclei by inducing nucleation at the first lower temperature and starting crystal growth;

d) uniformly spreading said initial nuclei into a bulk solution;

e) maintaining simultaneous and rapid growth of crystals from the nuclei of step d) at the first lower temperature for a predetermined length of time;

f) continuing the growth of said crystals to produce large, uniform and individual crystals for a predetermined length of time at a temperature that varies gradually from between a first lower temperature to a second lower temperature, wherein the second lower temperature is a temperature lower than the first lower temperature and further wherein the second lower temperature is an end temperature of crystallization; and

g) recovering the large, uniform and individual crystals.

In a preferred embodiment of the present invention, the induced nucleation of step c) is performed by mechanical impact of moving objects in a supersaturated solution and an addition of crystals just prior to isothermal crystallization. The step of maintaining and controlling a suitable nucleation rate is by adjusting a parameter selected from the group consisting of length of time, area, intensity and frequency of the mechanical impact of said moving objects in said supersaturated solution, mass amount, size, shape, other surface characters of the added crystals and a combination thereof, while keeping other parameters unchanged.

In a preferred embodiment of the present invention, the spreading of the initial nuclei of step d) is performed by means of intensive agitation in a short length of time without collision between crystals, and more preferably in a period of less than about 30 seconds.

In a most preferred embodiment of the present invention, the transition from said spreading of initial nuclei in step d) to said simultaneous and rapid growth of crystals in step e) is gradual without collision between crystals.

In a more preferred embodiment of the present invention, the growth of crystals in step f) is performed in a counter-current manner with an upward solution and downward crystals in a continuous operation. Additionally, step g) can further comprise the steps of:

i) separating low-density materials from a resulting crystal slurry using a cyclone to produce a mother solution containing produced crystals;

ii) separating the produced crystals from the mother solution;

iii) spray washing the produced crystals; and

iv) drying the wet crystals to obtain the large, uniform and individual crystals.

In a more preferred embodiment of the present invention, the method of producing crystals according to the present invention is for producing large, uniform and individual lactose monohydrate crystals from a concentrated whey permeate by means of evaporation. The concentration of total solids of the concentrated whey permeate is about 60% and the post-evaporation high temperature of the whey permeate is about 80° C. The first lower temperature is preferably from about 40° C. to about 55° C., and more preferably about 50° C. The simultaneously and rapidly growing crystals are uniformly suspended for a predetermined length of time preferably from about 45 minutes to about 60 minutes, and more preferably about 50 minutes. The second lower temperature is preferably from about 25° C. to about 30° C., for a predetermined length of time preferably from about 60 minutes to about 120 minutes. The total time of growth of the crystals is preferably from about 120 minutes to about 180 minutes.

The produced large, uniform and individual lactose monohydrate crystals preferably have an average size of at least tens of micrometers to hundreds of micrometers, preferably at least 80 micrometers, more preferably at least 150 micrometers and most preferably at 250 micrometers.

In another embodiment of the present invention, a system for generating crystals in a continuous operation manner is provided that includes:

a) a heat exchanger configured to accept and rapidly cool a concentrated permeate;

b) a nucleator configured to induce nucleation from the concentrated permeate;

c) a multifunctional crystallizer configured to assure isothermal crystal growth, maintain crystal growth in a predetermined temperature range that varies gradually from between a first lower temperature to a second lower temperature, classify crystals that are produced and collect the crystals; and

d) pumps and pipes configured to transport materials to and from the multifunctional crystallizer;

wherein the heat exchanger, the nucleator and the multifunctional crystallizer are connected together to generate the crystals.

In a preferred embodiment of the present invention, the heat exchanger is configured to cool a concentrated whey permeate.

In another preferred embodiment of the present invention, the nucleator includes metal rotating parts and fixed parts, and a contact area, wherein the rotating parts have an adjustable rotation speed and further wherein the rotating parts, the fixed parts and the contact area are arranged and configured to provide a specified contact intensity for the control of induced nucleation. The crystallizer further includes a central tube and a duct tube at an upper part for isothermal crystal growth, an upper cylinder and a lower cone body for crystal growth having a lower temperature than the temperature of the upper part, a hydraulic classification zone under the cone body, a crystal collector at a bottom of the crystallizer and a jacket surrounding the crystallizer for cooling.

### BRIEF DESCRIPTION OF THE DRAWINGS

FIG. 1 is an example of a process for refined lactose manufacture known in the art.

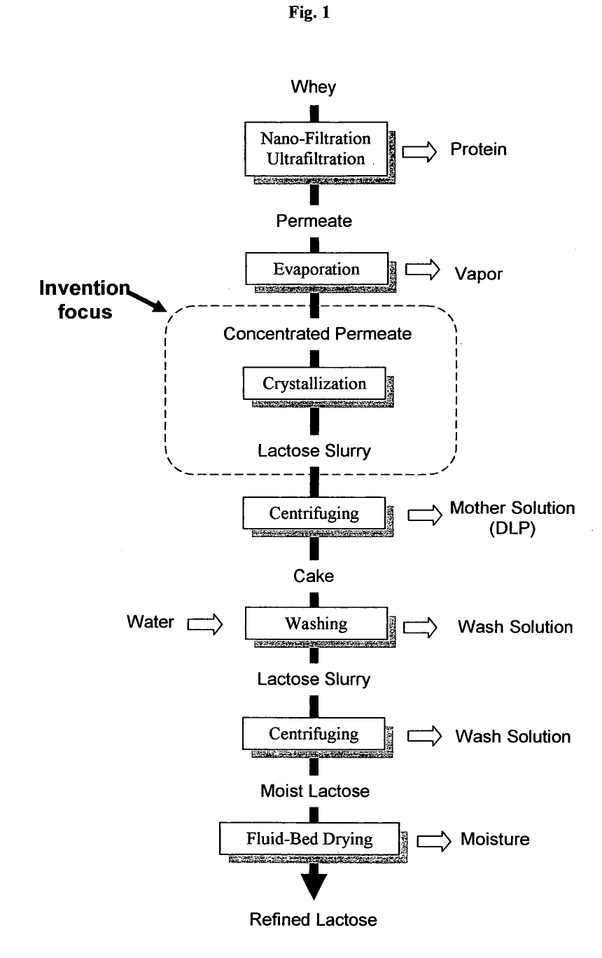

FIG. 2. shows the crystallization procedures and temperature profile for a process known in the art.

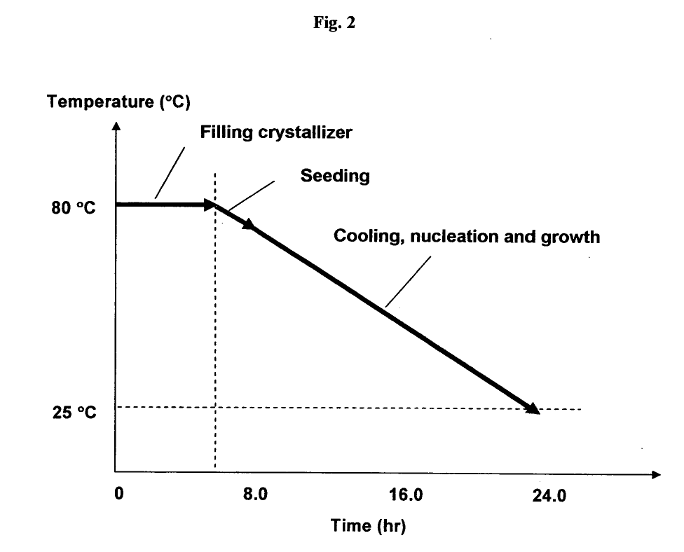

FIG. 3. shows a practical profile of the temperature plus concentration and the supersaturation of the solution in a process known in the art.

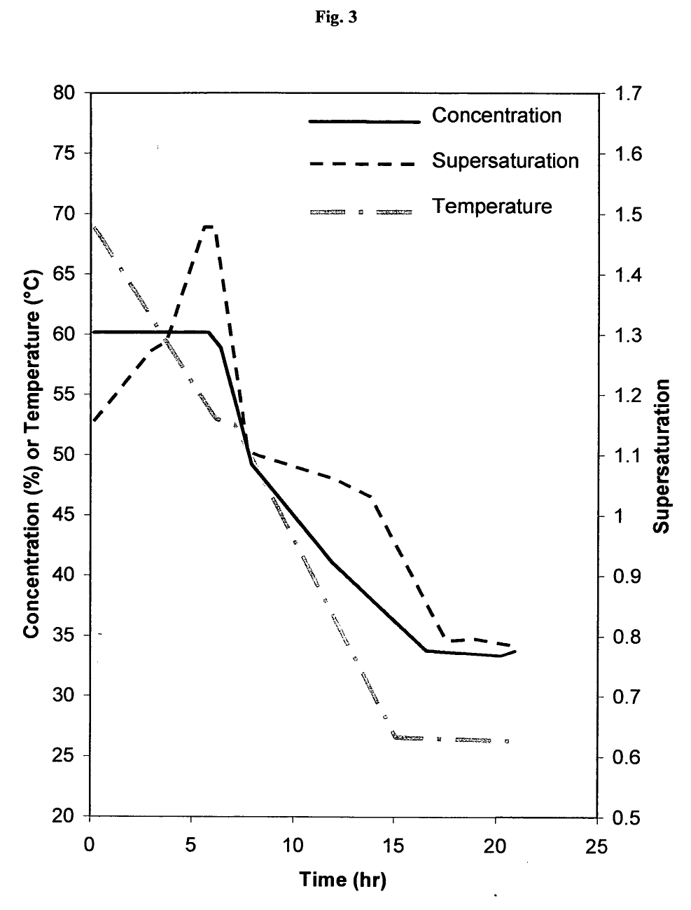

FIG. 4 shows an image of crystals produced in a crystallizer in a current commercial process known in the art.

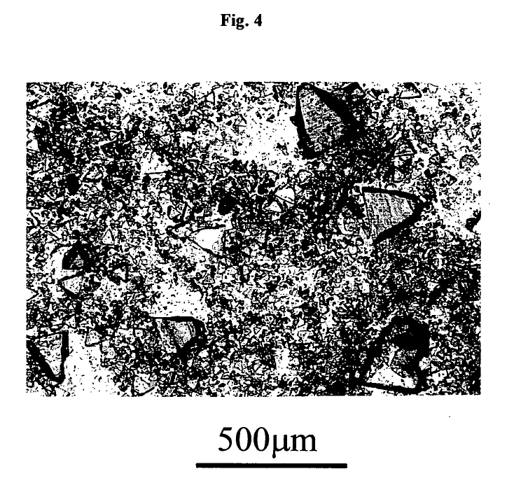

FIG. 5 shows the crystallization procedures and the temperature profile of the method of the present invention.

FIG. 6 shows the images of the crystals produced in a crystallizer in a method of the present invention from a lactose solution (A) and from the whey permeate (B).

FIG. 7 shows the mass crystallization rate for model lactose systems with different supersaturation levels by static crystallization at different temperatures.

FIG. 8 shows the linear growth rate of lactose crystals for model systems with different temperatures and concentrations.

FIG. 9 shows the yield during crystallization for a 60% lactose solution at different temperatures and with an agitation of 500 rpm/30 s for induced nucleation.

FIG. 10 shows the nucleation rate for model lactose systems at different temperatures and lactose concentrations and with an agitation of 500 rpm/30 s and the same seeding condition for induced nucleation.

FIG. 11 shows the effect of the contact duration on the crystal size distribution for inducing nucleation at 50° C., in a 59% solution and with crystallization for 2 hours according to the method of the present invention.

FIG. 12 shows the effect of the seed amount on the crystal size distribution for inducing nucleation at 50° C., in a 59% solution and with crystallization for 2 hours according to the method of the present invention.

FIG. 13 shows the repeatability of induced nucleation and subsequent crystal growth under the controlled operating conditions of 59% lactose solution, 0.025 g seed/100 g solution, agitation of 500 rpm/35 s for nucleation and agitation of 100 rpm for growth according to the method of the present invention.

FIG. 14 shows the optimal duration for contact induced nucleation at different temperatures for a pure aqueous lactose system with about 60% total solids according to the method of the present invention.

FIG. 15 shows a flowchart for a process in a continuous operation according to the system of the present invention.

FIG. 16 shows a schematic drawing of a crystallizer with a structure for continuous operation according to the system of the present invention.

FIG. 17 shows images of growing lactose crystals in whey permeate systems which show the principles applied to the methods of the present invention.

FIG. 18 shows images of lactose crystals obtained during the crystallization of the whey permeate concentrate according to the method of the present invention.

FIG. 19 shows an example of a nucleator that can be used in the methods of the present invention.

### DETAIL DESCRIPTION OF THE INVENTION

The present invention relates to methods of making crystals from raw materials. In a specific embodiment, the present invention relates to methods of improving crystal production from lactose monohydrate. Broadly, the methods of the present invention involve:

(A) Preparing a batch of a suitable number of nuclei per unit mass (g) of solution for growth based on 1) a batch of lactose nuclei that can be instantly generated by induction followed by simultaneous growth in a supersaturated solution, 2) the number of initial lactose nuclei by induced nucleation that can be quantitatively controlled, and 3) the induction process that is used such as seeding, contacting or a combination thereof. The suitable number of nuclei are based on these factors and other factors or conditions described herein.

(B) Providing optimal operating conditions (concentration, temperature, nuclei number, etc.) for a controlled induced nucleation, allowing crystals to grow at a maximum mass crystallization rate and a linear growth rate and completing the major crystallization in about 1 hour. The optimal operating conditions are based on the concepts that 1) induced nucleation behavior is controllable in a range of certain operating conditions, 2) the mass crystallization rate has an optimal region associated with certain operating conditions, and 3) the same operating conditions also result in a maximum linear growth rate.

(C) Controlling the initial nuclei number in addition to other conventional means (such as avoidance of strong agitation and avoidance of mechanical impact) to avoid secondary nucleation during crystal growth based on the concepts that 1) there is a suitable number of initial nuclei per unit mass of solution with which non-aggregate, large and relatively uniform crystals can be produced, 2) if the number of initial nuclei is greater than the premium number of initial nuclei, relatively uniform but small crystals can be produced, and 3) if the number of initial nuclei is less than the premium initial nuclei number, secondary nucleation will continuously occur and crystals with a wide crystal size distribution will be produced. The premium number of initial nuclei is the optimal number of nuclei for growing large, non-aggregated crystals according to the present invention. This number depends on the properties of the original solution such as the permeate, the concentration, the impurities and the crystallization temperature.

(D) Non-aggregated crystals instead of aggregated crystals are developed in order to eliminate entrainment of the mother solution and to facilitate separation by centrifugation. This process is based on the concepts that 1) crystal aggregates are the combinations of multiple crystals that are usually formed by the impact of rapidly growing crystals to each other, particularly, the small crystals in the initial growth phase after nuclei generation, or by the large difference in crystal size, 2) a gradual transition from the initial nuclei formation phase to the rapid growth phase, prevents the aggregation of crystals, and 3) a crystals/solution co-current operation in the crystal rapid growth phase wherein the flow of the solution and the flow of the crystals are in the same direction, is beneficial to the formation of non-aggregated crystals due to the elimination of the impact of the rapidly growing crystals to each other.

The inventors have surprisingly found that controlling the nucleation and crystal growth kinetics for lactose and whey permeate systems and the like, are the key components to improving lactose crystal production. This concept is applicable for other systems and growth of other types of crystals other than lactose. These crystals can be, but are not limited to, sodium chloride, sucrose, galactose, etc. All of these crystals have similar nucleation characteristics to those of lactose, such as, nucleation can be induced under certain high supersaturation level, the induced nucleation rate can be controlled, crystals can grow rapidly and simultaneously without accompanying secondary nucleation, the crystal size distribution is determined by the number of initial nuclei, etc. Therefore, the method and system according to the present invention can be used for these crystals.

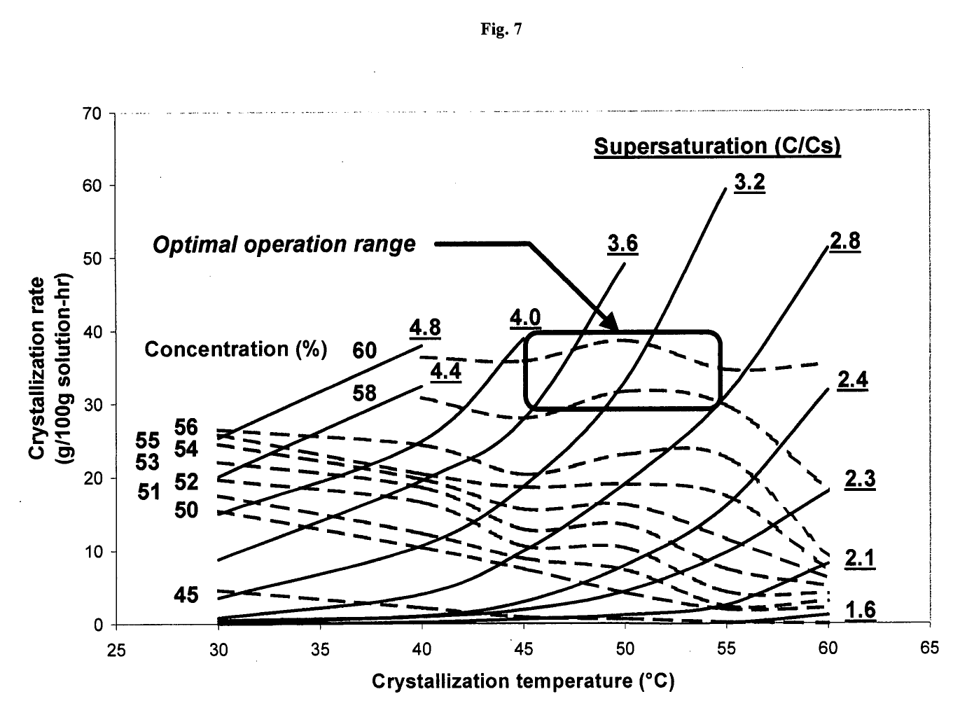

The static and dynamic crystallization of lactose monohydrate from solutions with a different concentration and at different temperatures was examined by the inventors of the present invention. The relationships between the average mass crystallization rate of lactose and the supersaturation levels/temperatures are shown in FIG. 7. FIG. 7 shows the crystallization rate in terms of mass increases as the supersaturation level increases. For systems with the same supersaturation level, yield increases as crystallization temperature increases. As the temperature increases, the solubility of α-lactose increases which makes the supersaturation level decrease. Also, the viscosity of a solution decreases to benefit the mass transfer. Additionally, the mutarotation balance between α-lactose and β-lactose shifts to benefit the conversion from β-lactose to α-lactose. Therefore, for systems with the same lactose concentration, the crystallization rate will be determined by the combined effects from all factors as shown by the black dashed lines in FIG. 7. It is revealed that lactose solutions with a higher concentration (e.g., ≧58%) can have a maximum crystallization rate only at a temperature of about 40° C. to about 55° C., and preferably about 50° C., as shown in the dashed rectangle area in FIG. 7.

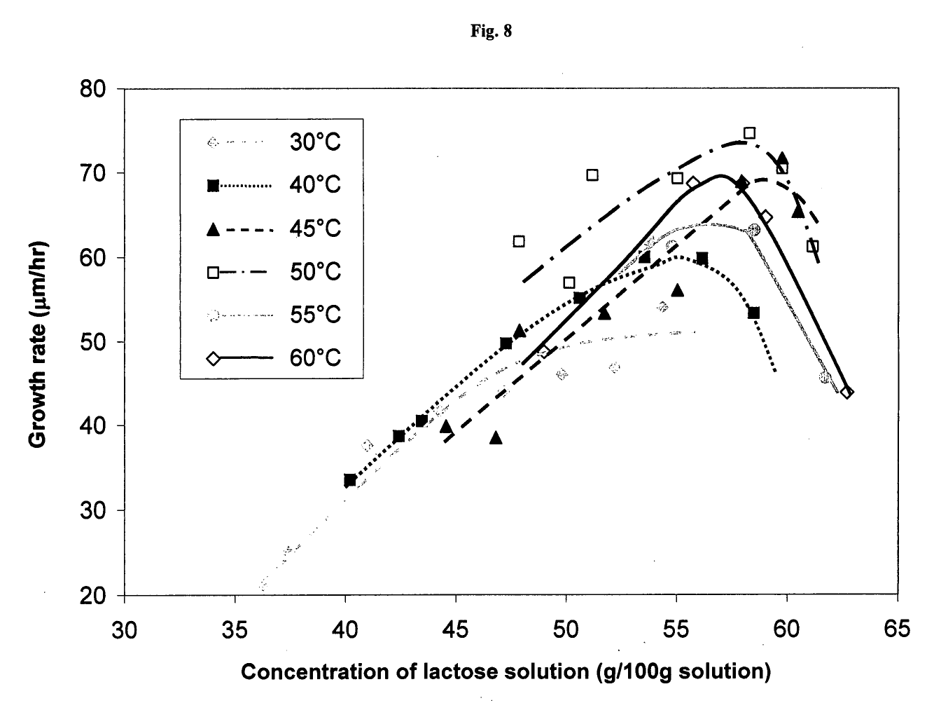

The crystallization rate in terms of linear growth (length increase per unit time) was also studied for model systems with different lactose concentrations at different temperatures and the results are shown in FIG. 8. In addition, the number of crystals and the associated total surface area of the crystals have a significant impact on the linear growth rate. Usually, driven by the same supersaturation level, a system with more crystals will have a lower linear growth rate. Thus, the number of crystals produced in a system is also affected by many factors. The trend lines in FIG. 8 show that within a certain temperature range, i.e., from about 40° C. to about 55° C., the linear growth rate has a curve from low to high and then from high to low as the lactose concentration increases. There is no simple trend of linear growth rate related to either temperature or lactose concentration. However, because of a combination of all of the factors, a system with a relatively high lactose concentration, i.e., at least 58% for a model system, with an optimal number of nuclei will have maximum linear growth rate at a temperature of about 40° C. to about 55° C., and preferably about 50° C. This is consistent with the results for mass crystallization rate as shown in FIG. 7. The optimal temperature of about 40° C. to about 55° C., and preferably about 50° C., is attributed from the relatively high supersaturation level (according to temperature, solubility and concentration), the higher conversion rate from β-lactose to α-lactose determined by mutarotation, the lower viscosity that is beneficial to mass transfer and the good nucleation controllability. With a controlled nucleation to generate an optimal number of nuclei, the growth of the lactose crystals can be driven relatively fast by a relatively high supersaturation.

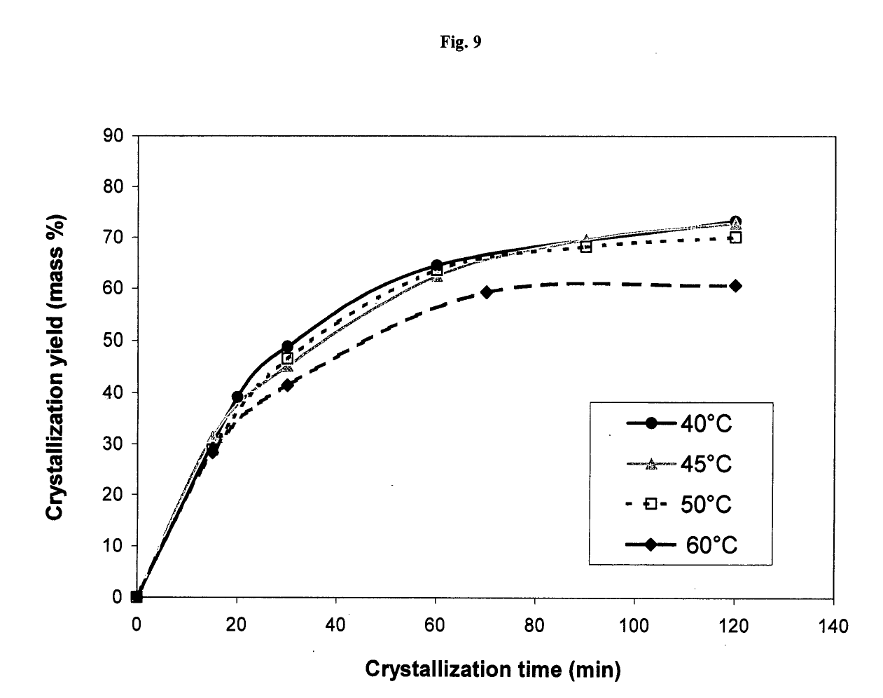

FIG. 9 shows the lactose crystal yield during crystallization for systems with a lactose concentration of 60% at different temperatures. The yield increases rapidly in the early phase, then its rate of increase slows until it reaches a maximum value. It can also be seen that crystal yield during crystallization at 50° C. is very close to those at 40° C. and 45° C. at a relatively higher level of yield. These results are also summarized in Table 2 for comparison. For example, at 50° C., the crystal yield reaches 46.6% (or 64.4% of the maximum) in a period of 30 minutes growth and the crystal yield reaches 70% (or 96.9% of the maximum) in a period of 60 minutes growth. These results indicate that most of the crystallization can be completed in a relatively short period of time (about 1 hour) and provide a basis for an efficient process.

TABLE 2
Crystal yield during crystallization of 60%
lactose solution at different temperatures

| Temperature (° C.) | 60 | 50 | 45 | 40 |
|---|---|---|---|---|
| Max. yield (%)* | 61.6 | 72.3 | 75.8 | 79.8 |
| 30-min. yield (%) | 41.4 | 46.6 | 45.2 | 48.9 |
| 30-min, yield/Max. yield (%) | 67.3 | 64.4 | 59.7 | 61.3 |
| 60-min. yield (%) | 60.7 | 70.0 | 72.8 | 73.2 |
| 60-min. yield/Max. yield (%) | 98.5 | 96.9 | 96.1 | 91.7 |

*possible maximum yield based on solubility

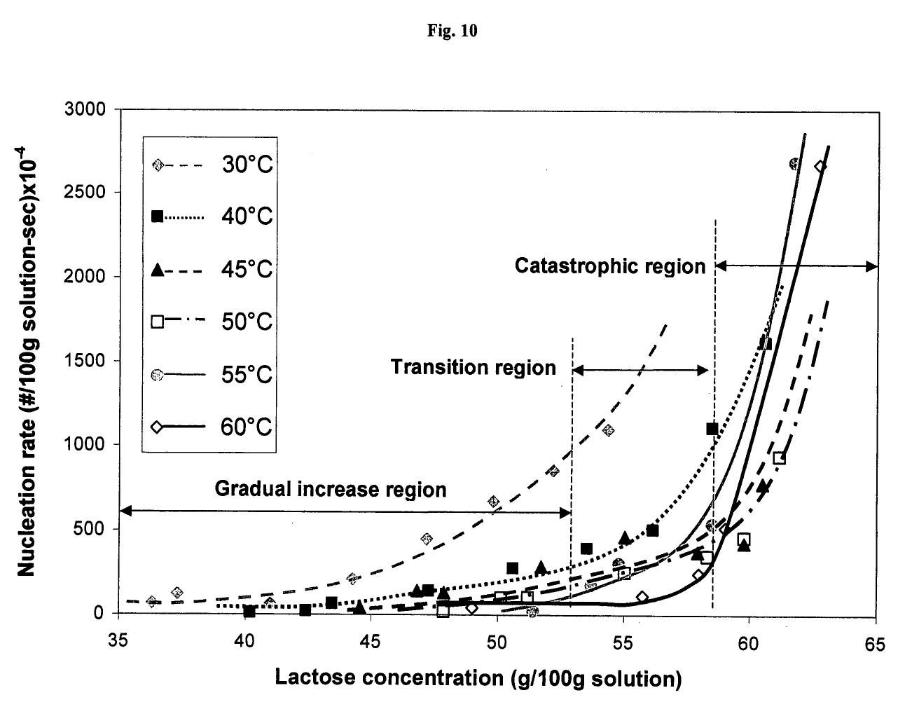

Nucleation is the most important phase for the crystallization operation. The rates of induced nucleation, or the number of generated initial nuclei per a certain solution mass (100 g of solution) and per unit time (second), i.e., # of initial nuclei/100 g solution-sec, for systems with different concentrations and at different temperatures (hence different supersaturation levels) are shown in FIG. 10. For systems with the same lactose concentration, the induced nucleation rate increases as the temperature decreases. At a certain temperature, systems with a higher lactose concentration will have a higher nucleation rate. The increase of nucleation rate in these systems is gradual when the lactose concentration is in a lower range but becomes very sharp when the lactose concentration is higher than 55% to 60%. In this catastrophic region, nucleation is very sensitive to the operating conditions and is hard to control. The methods of the present invention use the means of induction in order to control nucleation.

The inventors have surprisingly found that induced nucleation can be realized by seeding and/or object contacting in a highly supersaturated solution. It was found that at the moment that a supersaturated solution is contacted by seed crystals, formation of a large number (such as about 10,000,000 nuclei/100 g solution as seen in FIG. 10) of fine nuclei (called initial nuclei) with uniform size is triggered and once the seed crystals start growing, formation of new nuclei is very limited. At the same time, once the generated fine nuclei start rapidly growing, the number of crystals in the system remains basically unchanged, i.e., simultaneous growth of the crystals is reached. It was also found that the formation of a large number of initial nuclei with a uniform size can also be triggered by mechanical impact between objects. Generally, the number of initial nuclei that are produced by induced nucleation, is dependent on the supersaturation level (which is based on both the concentration and the temperature), the amount (mass and number), crystal size and shape of the seeds, the contact area, the contact frequency, the contact duration, etc. More specifically, for object contacting, the nucleation rate can be controlled by adjusting at least one of the following parameters: the length of time for nucleation, the contact area, the intensity of object contacting and the frequency of object contacting. While more than one of these parameters can be adjusted, it is preferred that only one parameter be adjusted while keeping the other parameters unchanged. For seeding, the nucleation rate can be controlled by adjusting at least one of the following parameters: the mass amount of the seed crystals, the size of the seed crystals, the shape of the seed crystals and other surface characteristics of the seed crystals. As with the object contacting, while more than one of these parameters can be adjusted, it is preferred that only one parameter be adjusted while keeping the other parameters unchanged. As mentioned herein, induced nucleation can be realized by seeding, object contacting or by seeding and object contacting. Preferably, induced nucleation is realized using only seeding or object contacting.

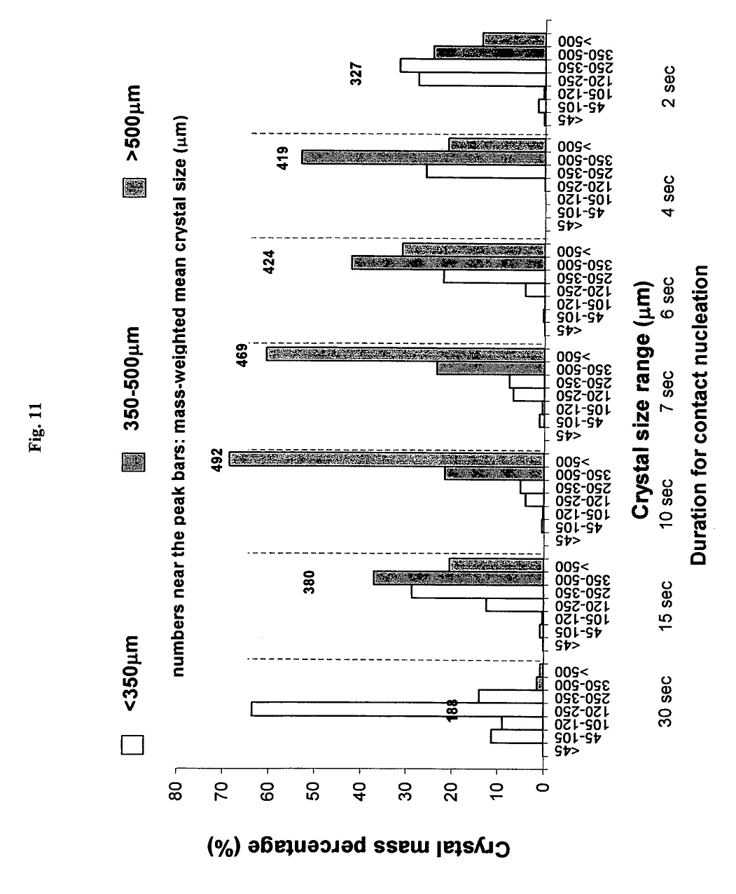

For contacting with a certain device having a known contact area, the adjustment of contact duration is an easy method for controlling induced nucleation. In the present invention, contact induction can be produced by using contacting a stainless steel spatula and an impeller of an agitator running at 500 rpm. The nucleation rate is controlled by altering the duration of contact. It was found that the contact duration of about 7 seconds to about 10 seconds resulted in a crystal product having a very good crystal size distribution (FIG. 11). This method of controlling the nucleation rate by using a different contact duration can be used for a batch operation because of the operating characteristics of the batch operation. However, in a continuous operation, a nucleator that is equipped with a contacting device and located before a crystallizer can be used to adjust one factor (frequency, contact area or intensity of contact).

The impact between the crystals and the wall of the crystallizer can cause secondary nucleation and the contact between the resulting rapidly growing crystals will lead to aggregated crystals. Once the initial nuclei are generated and start to quickly grow, secondary nucleation and aggregated crystals can be avoided by 1) immediately and uniformly dispersing the initial nuclei into the bulk solution quickly, 2) providing a gradual transition from the induced nucleation phase to the crystal growth phase in all conditions or parameters listed above, and 3) suspending the initial nuclei in the bulk without any impacting or contacting after nucleation induction.

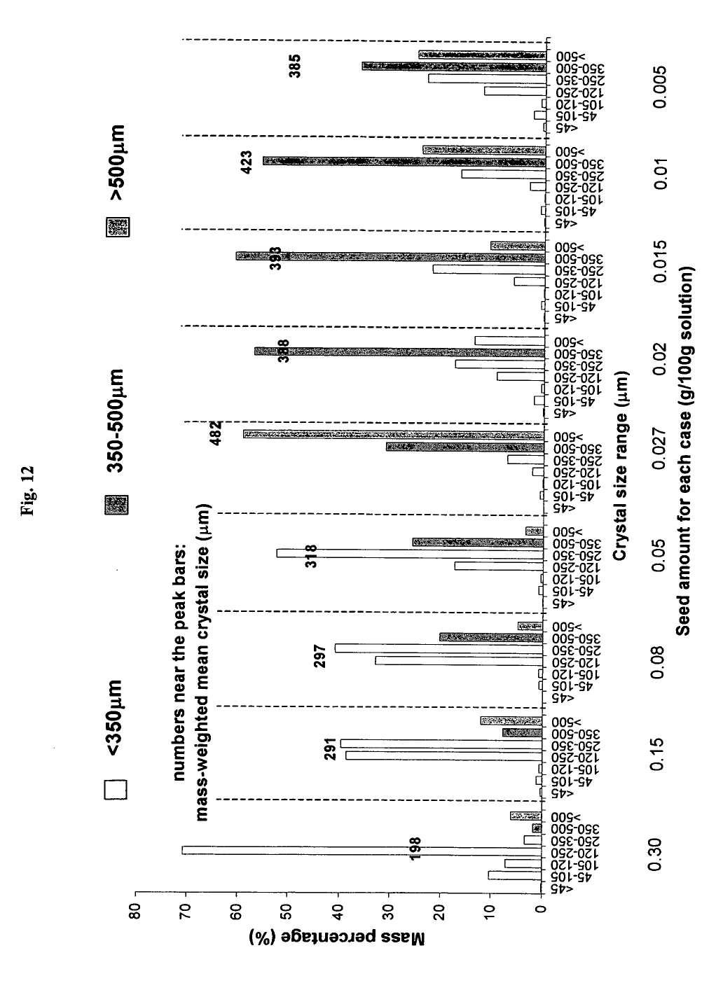

For seeding with crystals having a certain size range and surface characters, the adjustment of seed amount can be used for controlling induced nucleation. FIG. 12 shows a determination of the effect of the seed amount on crystal size distribution for induced nucleation, which are described herein.

Secondary nucleation is a common phenomenon that occurs when crystals already exist and grow. It is generally accepted that secondary nucleation is usually caused by impact between existing crystals and/or other objects (e.g., an agitator impeller or the crystallizer wall) in a supersaturated system. The inventors have surprisingly found that, instead of external factors such as mechanical impact, inherent reasons or factors, such as the number of nuclei, for secondary nucleation are more important. When the growth rate of existing crystals and the rate of change from clusters (orderly arranged molecules of crystalline material) to crystals in the supersaturated solution are out of balance, secondary nucleation will occur until a balance between the two rates is established. For example, if the number of existing crystals (usually generated from induced nucleation) is less, secondary nuclei will be generated much easier, because the existing crystals in this case consume limited lactose from solution and extra lactose clusters are available for the formation of nuclei. In this case, in addition to crystals developed from the initial nuclei, there are crystals developed from secondary nuclei, and the lasting time for secondary nucleation and the number of secondary nuclei increase as the number of initial nuclei decrease. If the number of existing crystals (usually generated from induced nucleation) is large and in a rapid growing status, they will consume a large amount of solute and leave no chance for secondary nucleation. However, if the existing crystals are not in rapid growth status, secondary nucleation still has a chance to occur due to the existence of a large amount of contact between the crystals. Therefore, a suitable number of existing crystals that are derived from an optimal number of initial nuclei by primary induced nucleation and that are in a fast growing status is necessary not only for the development of single crystals having uniformly large size, but is also necessary for the prevention of secondary nucleation. If the number of initial nuclei is greater than the optimal value, the average size of the developed crystals will tend to be smaller. If the number of initial nuclei is smaller than the optimal value, the crystal growth will be accompanied by a secondary nucleation and the crystal product will have a wide size distribution with a variation proportional to the difference in the number of initial nuclei that is between the actual number and optimal number. These surprising and significant observations were found by the inventors and described herein.

FIG. 11 and FIG. 12 show the mass percentage of crystal groups with different size ranges after 2-hour of crystallization using controlled induced nucleation by contact with a different duration and by seeding with a different seed amount, respectively. Results of 10 seconds contact duration (in FIG. 11) and results of 0.027 g/100 g solution seeding (in FIG. 12) show that most of the weight is contributed from crystals larger than 500 μm, some from crystals between 350 μm and 500 μm and little from small crystals. This indicates that there is a very good CSD with uniformly large crystals and a very limited number of secondary nuclei developing into small crystals. It also indicates that the induced nucleation is optimally controlled. However, the results represented by the cases on the left side (15 seconds or 30 seconds contact duration and 0.05 g/100 g, 0.08 g/100 g, 0.15 g/100 g or 0.30 g/100 g solution seeding) of the optimal conditions (10 seconds contact duration and 0.0027 g/100 g solution seeding), show that as the duration of contact or the seed amount increases, the number of formed initial nuclei in these systems increases such that the resulting crystals will have a relatively narrow size distribution but will have a smaller average size. This also indicates that, in terms of primary induced nucleation, these systems are over-nucleated. However, the results represented by the cases on the right side (7 seconds, 6 seconds, 4 seconds or 2 seconds contact duration and 0.02 g/100 g, 0.015 g/100 g, 0.01 g/100 g or 0.005 g/100 g solution seeding) of the optimal conditions, as the duration of contact or the seed amount decreases, the number of formed initial nuclei in these systems decreases. Although the initial nuclei in these systems can grow rapidly to develop into large crystals, the resulting crystal size distribution will gradually get wider, because their growth is always accompanied by secondary nucleation. Compared to the optimal case, the weight portion in these systems is contributed mostly from the smaller size crystals. Therefore, the CSD is not optimal due to the large number of small crystals that resulted from the large number of generated secondary nuclei (there is a low number of primary induced nuclei that do not consume enough lactose from the solution for their growth). This also indicates that, in terms of primary induced nucleation, these systems are under-nucleated.

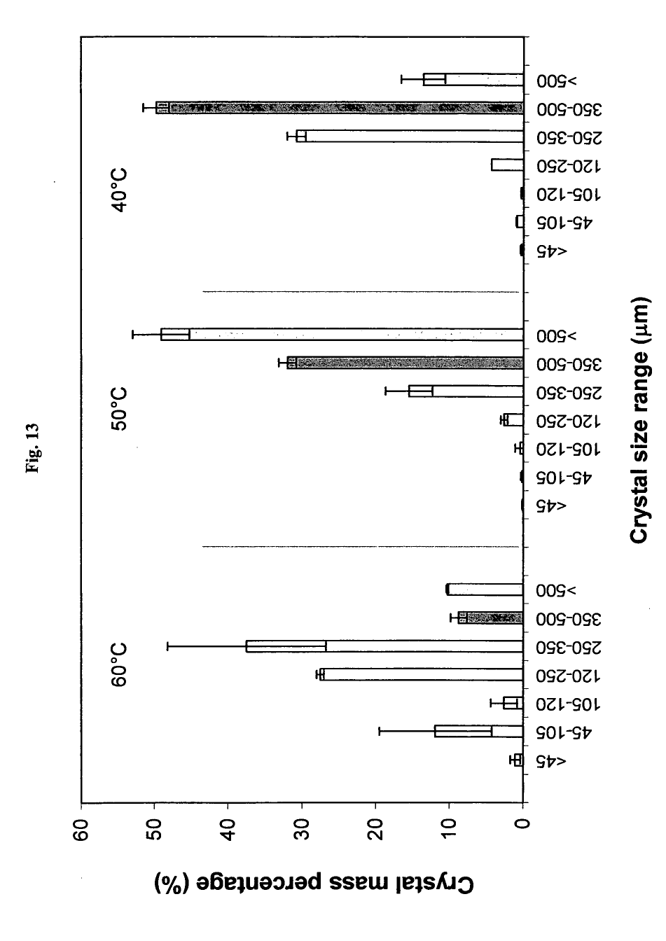

FIG. 13 shows the repeatability of induced nucleation and subsequent crystal growth under controlled conditions as indicated by reasonable standard deviations (The phrase “reasonable standard deviations” refers to the extent of variation and indicate how good the repeatability is. They are related to the high correlation coefficient and p-value lower than a (or higher confidence level) using correlation and ANOVA analysis). The results of induced nucleation are dependent on many factors. Thus, suitable operating parameters vary depending on the properties of whey permeate and the requirements of the crystals that are produced.

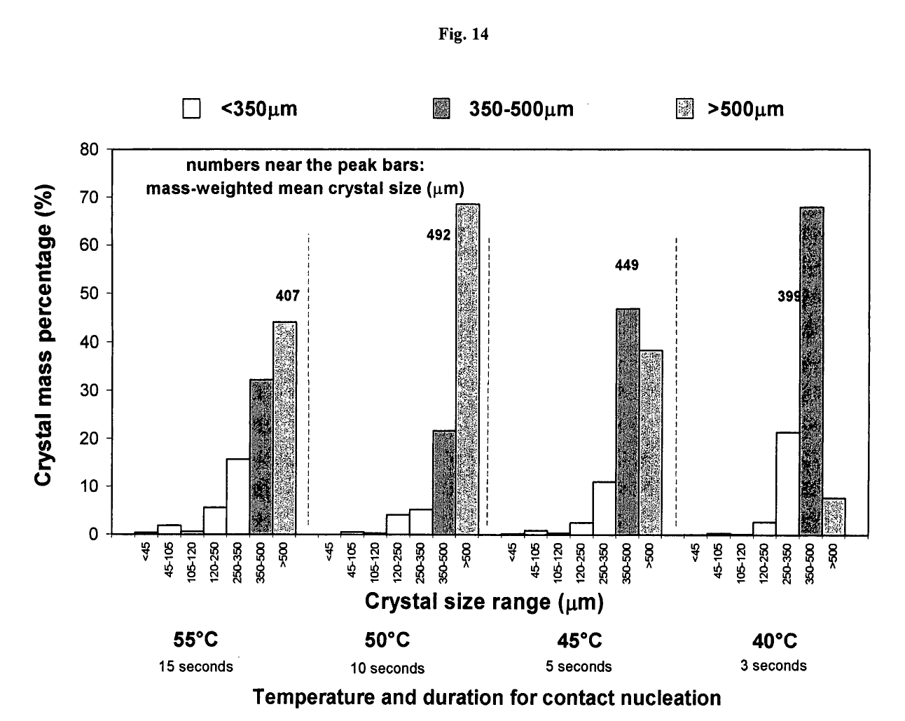

FIG. 14 shows examples for obtaining the optimal crystallization results by using contact nucleation at different temperatures. At 50° C., a 10-second contact duration is suitable, but at 55° C. and at 45° C., a 15-second and a 5-second contact duration is suitable, respectively, because the supersaturation levels are different at these different temperatures. For the actual systems, the suitable operating parameters must be determined according to their material composition, their concentration, the nucleation temperature, etc.

Therefore, the crystallization from a solution, such as that for lactose, can be optimized by controlling the primary induced nucleation to generate the appropriate number of nuclei, providing suitable conditions for rapid crystal growth and maintaining the secondary nucleation to a minimum level. The result is individual crystals with a uniform large size and a narrow crystal size distribution.

Although the methods according to the present invention are focused on the crystallization operation, the requirements of other operations, such as evaporation, are necessary to realize the invention. The invention will be understood more clearly from the following non-limiting representative examples as shown herein.

Production of large, uniform and individual lactose monohydrate crystals:

First, whey permeate is concentrated to a relatively high concentration (at least 58% or more, or preferably, about 60% total solids and 48-50% mass lactose) by, preferably, vacuum evaporation. This concentration is beneficial for controlled, induced nucleation.

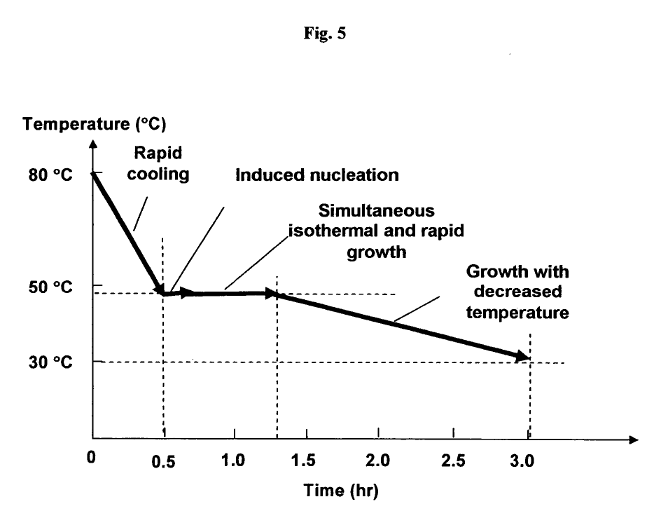

Preferably, the temperature of the concentrated permeate is about 80° C., the post-evaporation high temperature (FIG. 5). However, the range of temperature of the concentrated permeate can be from about 75° C. to about 90° C. The concentrated whey permeate is then transported via a pump into a heat exchanger where it is rapidly cooled (at least about 1° C./minute or higher) to the isothermal crystallization temperature or the first lower temperature of about 40° C. to about 55° C., preferably of about 50° C., depending on the sources of the whey permeate. This temperature range is beneficial for induced nucleation and isothermal crystal growth. During this cooling process, no formation of lactose nuclei occurs. Preferably, cooling is performed by the use of a high efficiency heat exchanger suitable for the concentrated whey permeate having specific physical and chemical properties. The resulting concentrated permeate has a relatively high supersaturation for lactose.

Next, a batch of initial nuclei is generated from the supersaturated solution at a suitable nucleation rate (nuclei number per unit solution and unit time or #/g-sec) by means of induced nucleation at a temperature that is the same as the isothermal crystallization temperature or the first lower temperature. The suitable nucleation rate is necessary for rapid growth of the crystals without a secondary nucleation and an aggregation of crystals. The means for induced nucleation can be object contacting or seeding. For object contacting, the suitable nucleation rate is controlled by adjusting one of the following parameters: the length of time for nucleation, the contact area, the intensity of object contacting and the frequency of object contacting. It is preferable that only one parameter is adjusted while keeping the other parameters unchanged. For seeding, the suitable nucleation rate is controlled by adjusting one of the following parameters: the mass amount of the seed crystals, the size of the seed crystals, the shape of the seed crystals and other surface characteristics of the seed crystals. Again, it is preferable that only one parameter is adjusted while keeping the other parameters unchanged.

The generated initial nuclei are immediately and uniformly spread into the bulk solution in a very short period of time (less than 30 seconds) without impact between the nuclei. The nuclei are also gradually transitioned from conditions for induced nucleation to conditions for fast growth. Next, the nuclei are suspended in a mildly dynamic environment (a transitional state between static/laminate and turbulent states and related to the Reynold Number, a dimensionless indicator of flow state) in order to continue the fast isothermal growth for a predetermined length of time (usually about 1 hour but can be more and depends on the nature and properties of the whey permeate) at a constant temperature of 40° C. to about 55° C., preferably about 50° C. (the isothermal crystallization temperature or the first lower temperature). These conditions are beneficial for the fast growth of the crystals and for the prevention of secondary nucleation or the prevention of the formation of crystal aggregates. This amount of time for crystal growth will yield most of the lactose crystals.

Crystal growth is continued for about another 1 to 2 hours during which the system temperature is linearly decreased gradually from the isothermal crystal growth temperature or first lower temperature to the end crystallization temperature or second lower temperature of about 25° C. to about 30° C., preferably about 25° C. This will assure the achievement of a high yield of the lactose crystal product. The total predetermined length of time for isothermal crystal growth and the crystal growth with a decreased temperature is from about 120 minutes to about 180 minutes.

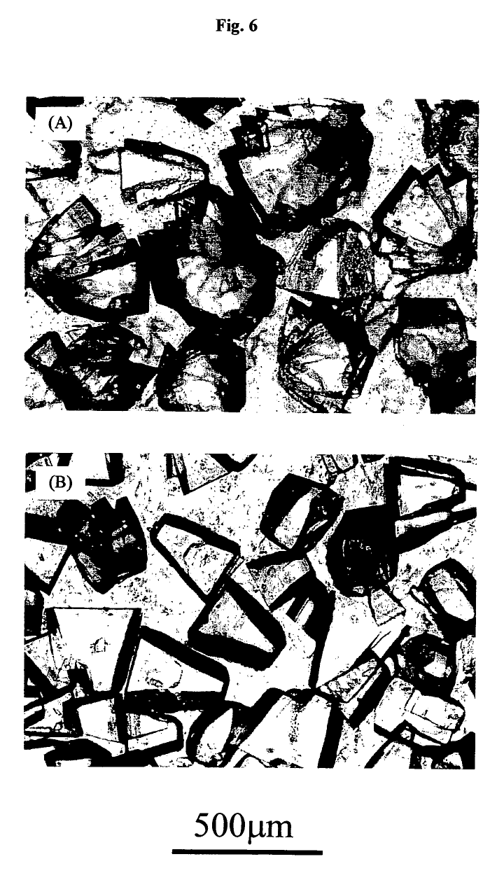

FIG. 6A shows an image of lactose crystals produced from a lactose solution according to the method of the present invention while FIG. 6B shows an image of lactose crystals produced from whey permeate according to the method of the present invention. The images show that the crystals produced from both lactose solution and whey permeate according to the method of the present invention are large, uniform and without secondary crystals.

Due to the complexity of the whey permeate composition, there may be other minute particles, such as organisms or minerals, with a lower density than the lactose crystals in the produced crystal slurry. These particles are first separated from the crystal slurry by using a cyclone. The result is a thickened crystal slurry that is then fed into a centrifuge for separation of the lactose crystals from the mother solution. Further treatments, such as washing and drying, are performed to obtain the final lactose crystal product.

The above-described method may be applied in a continuous operation, a batch operation or combinations thereof. However, there are certain differences in the performance between a continuous operation and a batch operation.

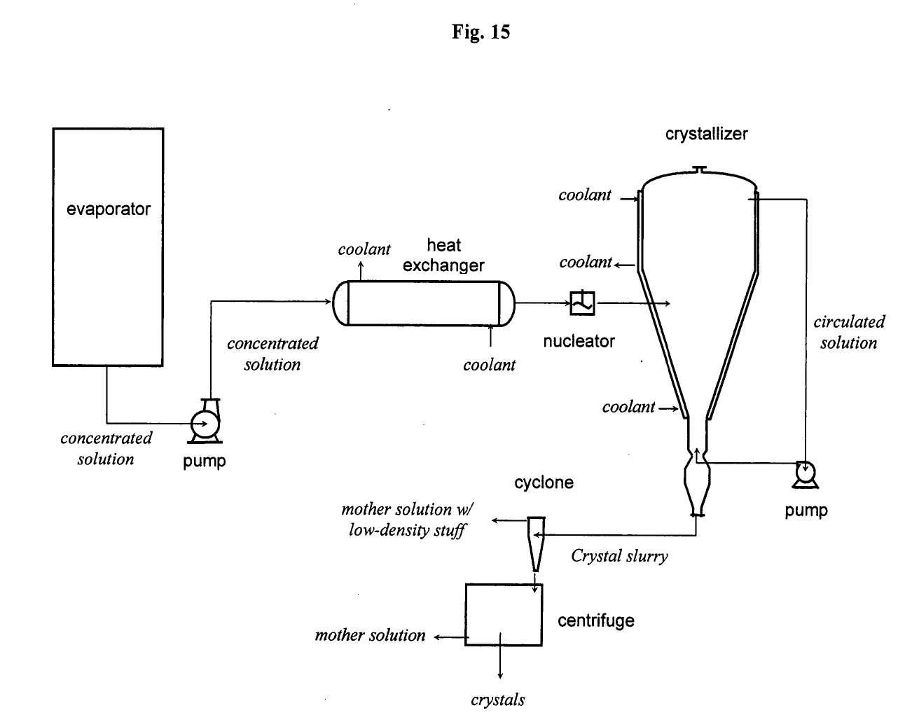

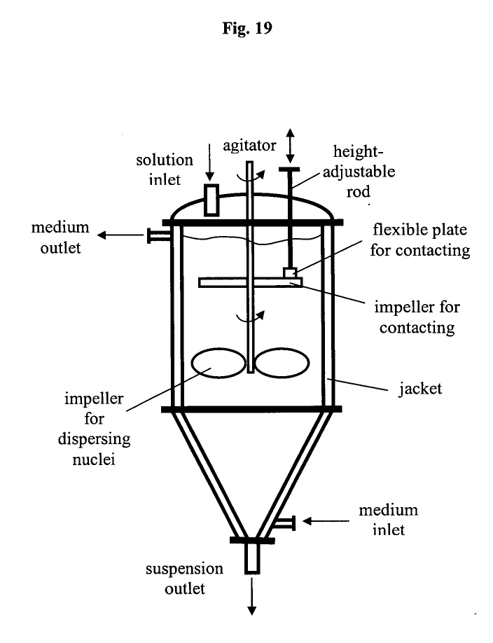

FIG. 15 shows a flow chart of a continuous operation according to the method and system of the present invention. The cooled, supersaturated permeate is transported into a separate equipment called a nucleator, where it is kept at a constant temperature, the isothermal crystal growth temperature or the first lower temperature. The nucleator is equipped with an object-contacting device that has a rotation mechanism, impacting parts and a function to adjust one of the above-mentioned parameters. The induction action of object contacting is continuous. The suitable operating parameters are determined by technological tests for a system with an actual material. For example, a continuous nucleator can be used. A continuous nucleator can, for example, comprise a small vessel with a jacket, an inlet, an outlet, an agitator with controlled speed, one or more impeller(s) for contacting and dispersing generated nuclei, and some semi-fixed (flexible) objects for enhancing contact with the impellers. An example of nucleator that can be used is shown in FIG. 19. The volume of the nucleator can be found based on V=W*T, where W is the flow rate (m³/minute), T is residence time and V is the volume (m³) of the nucleator. For the purposes of scale-up, different contact areas and/or agitation speeds (rpm) can be tested while keeping other factors unchanged.

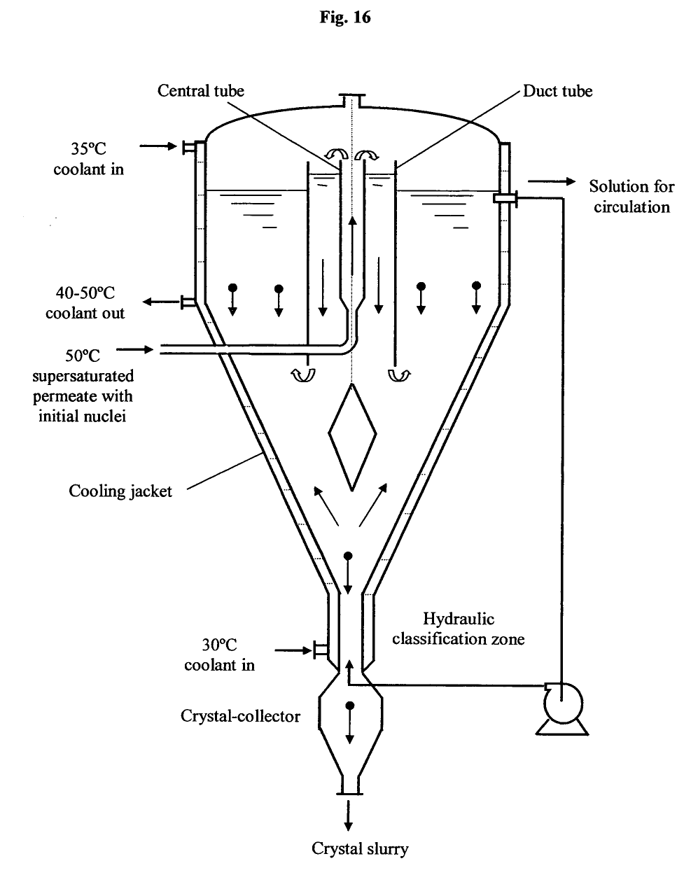

For a continuous operation that can fully take advantage of the present invention, a crystallizer as shown in FIG. 16 can be used. After the induced nucleation, the supersaturated permeate with the initial nuclei passes via a central tube of the crystallizer upwards and then via an annulus space between the central tube and the duct tube downwards as shown in FIG. 16, where the temperature is maintained at the isothermal crystallization temperature or the first lower temperature and the initial nuclei grow rapidly and simultaneously with a residence time of about 45 to 60 minutes. In the cone space of the crystallizer, the system will continue the crystal growth at lower temperatures from the isothermal crystallization temperature or the first lower temperature (cone top) to the end crystallization temperature or the second lower temperature (cone bottom) in order to reach the maximum yield. At the same time, the solution which may contain small crystals, flows up to the top of the crystallizer as the large crystals settle down. The solution is circulated by a pump into the crystallizer via the bottom of the hydraulic classification zone beneath the cone where crystals are classified by their size. From this, the crystals that are larger than a specified size and that are determined by the upward flow velocity of the circulating solution, enter the crystal collector. These crystals are then discharged. Due to the function of the circulating solution, a suspension bed is formed in the cone space and the annular space between the duct tube and the inner wall of the crystallizer. A cooling jacket around the crystallizer is used to maintain the temperature of the crystallizer.

For a batch operation, the major steps according to the method of the present invention take place in the crystallizer. The cooled, supersaturated permeate enters the crystallizer where it is kept at the isothermal crystallization temperature or the first lower temperature. Once the crystallizer is filled, contacting action will be applied using an agitator having impellers in order to impact other objects in the crystallizer. Seeding or seeding combined with object contacting may also be applied for the induced nucleation. Lactose crystal seeds having a suitable amount (depending on the nature of permeate and the characteristics of the seed crystals) are added into the crystallizer. The agitation is controlled at a predetermined intensity level for a predetermined length of time to generate a suitable amount and to immediately and uniformly spread the nuclei into the bulk solution. Induced nucleation is immediately followed by an isothermal, simultaneous and rapid crystal growth. For the batch operation, the temperature in the crystallizer is maintained at the isothermal crystallization temperature or the first lower temperature for about 1 hour and gentle agitation is applied to assure that the crystals are well suspended in the system. After the initial rapid growth, the system is gradually cooled from the isothermal crystallization temperature or the first lower temperature to the end crystallization temperature or the second lower temperature for crystal growth within about another 1 to 2 hours to reach a maximum crystal yield. In order to use the method according to the present invention and to improve the efficiency and the product quality, minor alterations may be needed for the existing batch crystallizers, to meet the requirements for induced nucleation.

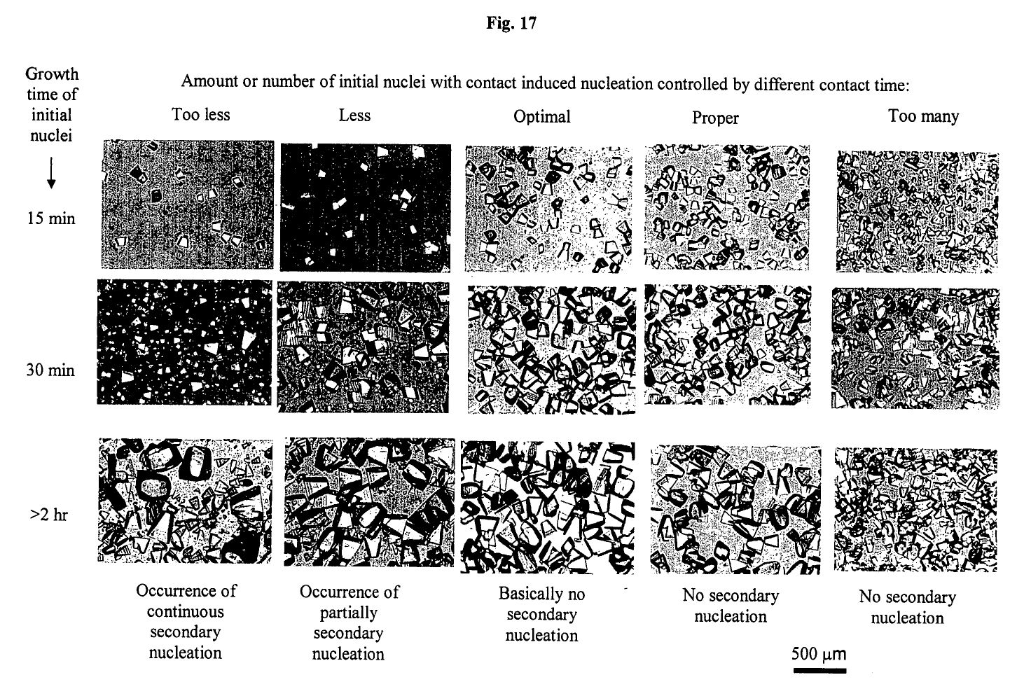

FIG. 17 shows that crystallization with permeate systems according to the method of the present invention was successfully employed for whey permeate systems. The total solids in these systems was 60%. All processing conditions were kept the same except the contacting time for induced nucleation in order to control the nucleation rate (amount or number of initial nuclei generated). The 5 columns from left to right in FIG. 17 represent 5 different studies. The images in the rows from top to bottom show crystals that are developed after a certain growth time—15, 30 and more than 120 minutes for each row. It can be seen that the obtained crystals have a larger size and a narrow size distribution if a suitable amount of initial nuclei are generated (Columns 3 and 4) because no secondary nucleation occurs. If the number of initial nuclei is not enough (Columns 1 and 2), the occurrence of secondary nucleation leads to a formation of many small crystals during the crystal growth, making the final product have a wide size distribution. If the nucleation rate is too high, the average size of developed crystals is small as shown in Column 5.

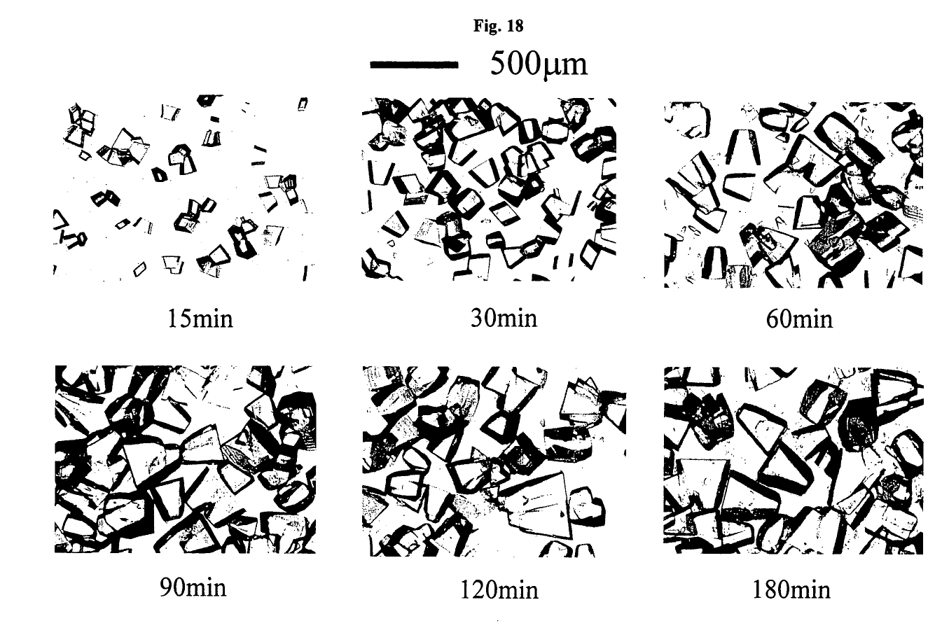

FIG. 18 shows images of lactose crystals obtained during the crystallization of the whey permeate concentrate according to the method of the present invention. It can be seen that the produced crystals are large, relatively uniform size with 95% of the crystals in a size range from 250 μm to 350 μm. There is a very limited number of small crystals which indicates that the initial nuclei number was successfully controlled and secondary nucleation was successfully prevented.

Changes can be made to the composition of the permeate, operating parameters as indicated above, the arrangement of the method and the system of the present invention without departing from the concept and scope of the invention as defined in the following claims:

What is claimed is:

1. A method for making large, uniform and individual crystals from aqueous solutions, said method comprising the steps of:
a) obtaining a concentrated aqueous solution by means of evaporation;
b) rapidly cooling said solution from a post-evaporation high temperature to a first lower temperature, wherein said first lower temperature is lower than the post-evaporation high temperature and further wherein said first lower temperature is an isothermal crystallization temperature of said solution;
c) generating a batch of initial nuclei by inducing nucleation at said first lower temperature and starting crystal growth;
d) uniformly spreading said initial nuclei into a bulk solution;
e) maintaining simultaneous and rapid growth of crystals from said nuclei of step d) at said first lower temperature for a predetermined length of time;
f) continuing the growth of said crystals to produce large, uniform and individual crystals for a predetermined length of time at a temperature that varies gradually from between a first lower temperature to a second lower temperature, wherein said second lower temperature is a temperature lower than the first lower temperature and further wherein said second lower temperature is an end temperature of crystallization; and
g) recovering said large, uniform and individual crystals.

2. The method of claim 1, wherein said crystals are made in an operation selected from the group consisting of a batch operation, a continuation operation and a combination thereof.

3. The method of claim 1, wherein the induced nucleation of step c) is performed just prior to isothermal crystallization.

4. The method of claim 1, wherein the induced nucleation of step c) is by mechanical impact of moving objects in a supersaturated solution.

5. The method of claim 1, wherein the induced nucleation of step c) is generated by an addition of crystals.

6. The method of claim 4, further comprising the step of maintaining and controlling a suitable nucleation rate by adjusting a parameter selected from the group consisting of length of time, area, intensity and frequency of the mechanical impact of said moving objects in said supersaturated solution and a combination thereof, while keeping other parameters unchanged.

7. The method of making crystals of claim 5, further comprising the step of maintaining and controlling a suitable nucleation rate by adjusting a parameter selected from the group consisting of mass amount, size, shape, other surface characters of the added crystals and a combination thereof, while keeping other parameters unchanged.

8. The method of claim 1, wherein the spreading of the initial nuclei of step d) is performed by means of intensive agitation in a short length of time without collision between crystals.

9. The method of claim 8, wherein the spreading of the initial nuclei in step d) is performed in less than about 30 seconds.

10. The method of claim 1, wherein the transition from said spreading of initial nuclei in step
d) to said simultaneous and rapid growth of crystals in step e) is gradual without collision between crystals.

11. The method of claim 1, wherein the growth of crystals in step f) is performed in a counter-current manner with an upward solution and downward crystals in a continuous operation.

12. The method of claim 1, wherein said crystals are produced having a narrow crystal size distribution.

13. The method of claim 1, wherein step g) comprises the steps of:
i) separating low-density materials from a resulting crystal slurry using a cyclone to produce a mother solution containing produced crystals;
ii) separating the produced crystals from the mother solution;
iii) spray washing the produced crystals; and
iv) drying the wet crystals to obtain the large, uniform and individual crystals.

14. A method for making large, uniform and individual lactose monohydrate crystals from whey permeate, said method comprising the steps of:
a) obtaining a concentrated whey permeate by means of evaporation;
b) rapidly cooling said permeate from a post-evaporation high temperature to a first lower temperature, wherein said first lower temperature is lower than the post-evaporation high temperature and further wherein said first lower temperature is a isothermal crystallization temperature of said permeate;
c) generating a batch of initial nuclei by inducing nucleation at said first lower temperature and starting crystal growth;
d) uniformly spreading said initial nuclei into a bulk solution;
e) maintaining simultaneous and rapid growth of crystals from said nuclei of step d) at said first lower temperature for a predetermined length of time;
f) continuing the growth of said crystals to produce large, uniform and individual crystals for a predetermined length of time at a temperature that varies gradually from between a first lower temperature to a second lower temperature, wherein said second lower temperature is a temperature lower than the first lower temperature and further wherein said second lower temperature is an end temperature of crystallization; and
g) recovering said large, uniform and individual lactose monohydrate crystals.

15. The method of claim 14, wherein said lactose monohydrate crystals are made in an operation selected from the group consisting of a batch operation, a continuation operation and a combination thereof.

16. The method of claim 14, wherein a concentration of total solids of said concentrated whey permeate of step a) is about 60%.

17. The method of claim 14, wherein said post-evaporation high temperature is about 80° C.

18. The method of claim 14, wherein said first lower temperature is from about 40° C. to about 55° C.

19. The method of claim 18, wherein said first lower temperature is about 50° C.

20. The method of claim 14, wherein the induced nucleation of step c) is performed just prior to isothermal crystallization.

21. The method of claim 14, wherein the induced nucleation of step c) is by mechanical impact of moving objects in a supersaturated solution.

22. The method of claim 14, wherein the induced nucleation of step c) is generated by an addition of lactose monohydrate crystals.

23. The method of claim 21, further comprising the step of maintaining and controlling a suitable nucleation rate by adjusting a parameter selected from the group consisting of length of time, area, intensity and frequency of the mechanical impact of said moving objects in said supersaturated solution and a combination thereof, while keeping other parameters unchanged.

24. The method of claim 22, further comprising the step of maintaining and controlling a suitable nucleation rate by adjusting a parameter selected from the group consisting of mass amount, size, shape, other surface characters of the added lactose monohydrate crystals and a combination thereof, while keeping other parameters unchanged.

25. The method of claim 14, wherein the spreading of the initial nuclei of step d) is performed by means of intensive agitation in a short length of time without collision between crystals.

26. The method of claim 25, wherein the spreading of the initial nuclei in step d) is performed in less than about 30 seconds.

27. The method of claim 14, wherein the simultaneously and rapidly growing crystals of step
e) are uniformly suspended and said predetermined length of time of step e) is from about 45 minutes to about 60 minutes.

28. The method of claim 27, wherein said predetermined length of time in step e) is about 50 minutes.

29. The method of claim 14, wherein the transition from said spreading of initial nuclei in step d) to said simultaneous and rapid growth of crystals in step e) is gradual without collision between crystals.

30. The method of claim 14, wherein said second lower temperature in step f) is from about 25° C. to about 30° C., and said predetermined length of time is from about 60 minutes to about 120 minutes.

31. The method of claim 14, wherein the growth of crystals in step f) is performed in a counter-current manner with an upward solution and downward crystals in a continuous operation.

32. The method of claim 14, wherein said predetermined length of time of step e) and said predetermined length of time of step f) total from about 120 minutes to about 180 minutes.

33. The method of claim 14, wherein said crystals are produced having a narrow crystal size distribution.

34. The method of claim 14, wherein step g) comprises the steps of:
i) separating low-density materials from a resulting crystal slurry using a cyclone to produce a mother solution containing produced crystals;
ii) separating the produced crystals from the mother solution;
iii) spray washing the produced crystals; and
iv) drying the wet crystals to obtain the large, uniform and individual lactose monohydrate crystals.

35. The method of claim 14, wherein said large, uniform and individual lactose monohydrate crystals have an average size of at least 150 micrometer.

36. The method of claim 14, wherein said large, uniform and individual lactose monohydrate crystals have an average size of at least tens of micrometers to hundreds of micrometers.

37. A system for generating crystals in a continuous operation manner, said system comprising:
a) a heat exchanger configured to accept and rapidly cool a concentrated permeate;
b) a nucleator configured to induce nucleation from said concentrated permeate;
c) a multifunctional crystallizer configured to assure isothermal crystal growth, maintain crystal growth in a predetermined temperature range that varies gradually from between a first lower temperature to a second lower temperature, classify crystals that are produced and collect said crystals; and
d) pumps and pipes configured to transport materials to and from said multifunctional crystallizer;
wherein said heat exchanger, said nucleator and said multifunctional crystallizer are connected together to generate said crystals.

38. The system of claim 37, wherein said heat exchanger is configured to cool a concentrated whey permeate.

39. The system for generating crystals of claim 37, wherein said nucleator comprises metal rotating parts and fixed parts, and a contact area; and
wherein said rotating parts have an adjustable rotation speed and further wherein said rotating parts, said fixed parts and said contact area are arranged and configured to provide a specified contact intensity for control of induced nucleation.

40. The system of claim 37, wherein said crystallizer comprises a central tube and a duct tube at an upper part for isothermal crystal growth, an upper cylinder and a lower cone body for crystal growth having a lower temperature than the temperature of said upper part, a hydraulic classification zone under said cone body, a crystal collector at a bottom of said crystallizer and a jacket surrounding said crystallizer for cooling.

41. The system of claim 37, wherein the system is configured for growing lactose monohydrate crystals.
